(19) United States
(12) Patent Application Publication
(10) Pub. No.: US 2022/0062664 A1
(43) Pub. Date: Mar. 3, 2022

(54) Potential Feedback Human Breath Filtration Device

(71) Applicant: Peter S. Yuen, San Jose, CA (US)

(72) Inventor: Peter S. Yuen, San Jose, CA (US)

(21) Appl. No.: 17/007,426

(22) Filed: Aug. 31, 2020

(51) Int. Cl. A62B 7/10; B01D 46/00; B03C 3/011; B03C 3/08; B03C 3/41; A61F 9/02; A61L 9/20; A61L 9/22; A62B 18/02; A62B 23/02; A62B 18/08; A62B 18/04; A62B 9/00

(52) U.S. Cl. CPC A62B 7/10; A61L 2209/14; B01D 46/0032; B03C 3/011; B03C 3/08; B03C 3/41; A61F 9/02; A61L 9/20; A61L 9/22; A62B 18/025; A62B 23/02; A62B 18/08; A62B 18/084; A62B 18/02; A62B 18/045; A62B 9/00; B01D 2279/40; B01D 2279/65; B03C 2201/06; A61L 2209/12; B01D 46/0028

(57) ABSTRACT

A DC power source operating portable human body carrying potential feedback electronic human breath filtration device is an electronic nose mask with directional ionic air filtration control and is the most ideal alternative to conventional filter paper type nose mask. It utilizes potential feedback electronic ionization technique and electrostatic field to remove air borne matters from human inhalation and exhalation breath. It has UV germicidal function and a micro electric fan to provide positive air pressure. It utilizes electromechanical potential feedback technology to improve the rate of ionization at lower voltage level to minimize the generation of Ozone; a moisture collection feature to absorb the condensation from the user's breath. It is light weight and connected to a pocket size control system via a connection cable and has an ionization flux sensing device for user to verify the present of the ionization.

### FIELD OF INVENTION

The present invention relates to positive air pressure respiratory filtration nose mask with electronic air filtration system and UV germicidal function for human breath, and more particularly, a filtration device for both inhalation and exhalation breaths. The new system is generic to function with and without a fan; and generic to be incorporated to a face mask, a helmet and a hood covering the user's head.

### BACKGROUND OF THE INVENTION

Nose mask has been widely used in all kinds of industries from medical to industrial; from field works to home cleaning; and in many different occasions whenever filtration of inhaling air is necessary. Usually the filter materials are of paper or fiber properties. The basic mechanism is using the human inhalation action as basic air suction driving force to suck the air through the filter media and stop all particles which is larger than the pores of the filtration media. It becomes very uncomfortable when someone wears the nose mask for a long time continuously and it is even worse if the user is kind of weak or having asthma or breathing difficulties. It is more obvious when during with PM2.5 filtration in highly polluted environment.

Secondly, the filtration function is usually less efficient during the exhalation because the exhaust air tends to leak through the edges along the users' face rather than through the filter media.

Thirdly, the air passage resistance of the better filtration media is always higher and tougher to inhale through it.

Fourthly, the power consumption of good air purification system is usually very high. It is good to have low power consumption system that can utilize USB 5 VDC power source to operate the system since USB 5 VDC power source is commonly available in most public transportation system like airplanes, buses, trains and ferries; as well as mobile phone backup power banks.

Ionization air filtration technique does help to solve all these issues. However, there is lot of room for improvement. Especially when ions are generated, they scatter and shoot off randomly. Experiments show that if the ions generated are concentrated at area along the airflow passage with control over the ions travelling direction, the overall air filtration efficiency magnifies.

In order to control the concentration of ions in the airflow passage, an electro-mechanical feedback system can be used to shape out the area where the concentration of ions to be located and the amount of concentration of ions can be determined to provide optimal ionization air purification results.

Thus, there is a need for a high efficiency inhalation and exhalation filtration with feedback system that does not exert breathing resistance to users during the normal breathing process. This filtration system shall be able to remove most of the contaminant in the air including airborne particles and substances. Since the

airborne substances collected by the filtration system may include bacteria and viruses, it is appropriate to incorporate a germicidal system to sterilize these organisms collected. Ultraviolet (UV) radiation system with wavelength between 200 Å to 400 Å is to be incorporated into the system to perform the germicidal sterilization of the collected organism. Light Emitting Diode with wavelength shorter than 440 Å is to be used for both UV radiation generation and as a heat source to minimize moisture condensation. A brushless DC motor driven impeller device is also incorporated into the system as a mean to balance the air resistance during the inhalation process of the user such that user will feel breathing through the filtration system is even easier when there is positive air pressure supplied by the mask. This brushless DC motor driven impeller device improves air circulation inside the mask chamber and helps to reduce moisture condensation as well. Regular electrical fan is made of brush motor and will generate carbon dust during operation. Brushless DC motor driven impeller device is without contacting brushes inside the motor and does not generate carbon dust.

Unlike the mechanical media filtration, ionic air filtration cannot be easily detected by users to know if ionization is really occurring and the mask is functioning as expected. An ionization flux detection device is needed to be provided to users such that they can easily verify the functionality of the ionization in the mask.

The whole system shall be light enough for users to feel comfortable if wearing for extended time. It shall be very efficient in power consumption such that small consumer electronic type battery pack can support operation of the system for over an extended period. It can also operate with power input from an USB 5 VDC power source such that user can be safe to sit close to each other since it is an individual air purification system directly providing purified air to the nose of the user. Easiness to clean and cost effective are also critical.

This is a generic system that can be adapted to respirator mask, face mask, hood and helmet to provide air purification to said devices.

Furthermore, the filtration process shall be as efficient during both inhalation and exhalation such that if a patient is the user; the bacteria or viruses from the user's breath will not get to outside ambient environment without passing through a purification process.

The present invention provides such an inhalation and exhalation filtration system nose mask. This nose mask is generic to be adapted to face mask, hood mask and helmet with mask. Users can also easily verify the presence of the ionization in the system.

### CROSS REFERENCE TO RELATED APPLICATIONS

| Field of Search | |
|---|---|
| International Class: | A62B 23/00, 7/10 |
| US Class | 128/200.24; 96/29, 54, 69, 71, 72, 75, |
| | 78, 97, 98, 100 |

| | U.S. Patent Documents |
|---|---|
| 4549887 | Oct. 29, 1985 Joannou 96/58 |
| | This is not a human breathe cleaning device. |
| 5042997 | Aug. 27, 1991 Rhodes 96/18 |
| | This is not a human body carrying electronic breath |
| | filtering mask. |
| 5232478 | Aug. 3, 1993 Farris 96/26 |
| | This is not a human body carrying electronic breath |
| | filtering mask. |
| 5573577 | Nov. 12, 1996 Joannou 96/66 |
| | This is not a human body carrying electronic breath |
| | filtering mask. |
| 5690720 | Nov. 25, 1995 Spero 96/26 |
| | This is not a human body carrying electronic breath |
| | filtering mask. |
| 5846302 | Dec. 8, 1998 Putro 96/66 |
| | This is not a human body carrying electronic breath |
| | filtering mask. |
| 6245132 | Jun. 12, 2001 Feldman 96/28 |
| | This is not a human body carrying electronic breath |
| | filtering mask. |
| 6497754 | Dec. 24, 2002 Joannou 96/67 |
| | This is not a human body carrying electronic breath |
| | filtering mask. |
| 7392806 | Jul. 1, 2008 Yuen 128/205.27 |
| | Electronic human breath filtration device. This is |
| | my prior patent. |

### SUMMARY OF THE INVENTION

A Potential Feedback Human Breath Filtration Device is a human wearable light weight nose mask equipped with an absolute miniature electronic filtration with electro-mechanical potential feedback system with positive pressure air flow to the user. The potential feedback system will increase the rate of ionization occur without raising the electrical negatively charged voltage significantly. This will reduce the amount of Ozone generation with respect to original ionization system without the potential feedback system while producing the equivalent quantity of ions. This device filters dust, smoke, airborne substances, odor and PM2.5 from entering the user's body through inhalation.

This is a generic system that can be adapted to respirator mask, face mask, hood mask and helmet to provide air purification to said devices.

The unique feature of this invention is to provide a control directional and concentration ions of electronic air filtration device with sterilization function to any organism captured by the device. The user can breathe through this filtration device without requiring extra effort as compare to sucking/breathing heavily through convention paper filter mask since a positive pressure is provided by the system. Users can also easily verify the presence of the ionization in the system with the ionic flux sensing device. For users' convenience, this invention can also use USB 5 VDC power source as power input to operate since USB 5 VDC power source is commonly available in most public transportation system like airplanes, buses, trains and ferries. Furthermore, common backup power banks for mobile phone user also equipped with USB 5 VDC power outlet and will be very convenient as power source to the said mask.

It is an object of this present invention to provide a very compact multiple stages element filtration system mounted on a nose mask and utilizing small consumer electronic size battery as power source to operate this high voltage ionic filtration system as well as electrostatic filtration system. It relies on the human breath as the air flow source to move the air stream through the multiple stages filtration system during the inhalation and exhalation processes with help of a small electric fan to add positive pressure against the small airflow resistance that generated along the air passage.

Other features and advantages of the invention will appear from the following description in which the preferred embodiments have been set forth in detail, in conjunction with the accompanying drawings.

### BRIEF DESCRIPTION OF THE DRAWINGS

Ultraviolet radiation is stated as UV ray in the following portion of the document and similarly for the following terms:

Ultraviolet is stated as UV.
Millimeter is stated as mm.
Light emitting diode is stated as LED.
Universal Serial Bus is stated as USB.
PM2.5 refers to atmospheric particulate matter (PM) that have a diameter of less than 2.5 micrometers.

FIG. 1 is the overall diagram of the Potential Feedback Human Breath Filtration Device. It depicts a portion of the sectioned nose mask, a portion of the sectioned multi-stages electronic filter, a portion of the sectioned front pre-filter system, a portion of the rear anti-UV ray leakage cover system with an electric fan, the USB power conversion cable, the ionization flux sensing device, the electronic control box, the connecting cable and a user wearing the device to demonstrate the relative usage of the system according to present invention.

FIG. 2 illustrates the isometric front view of the filtration system with the contoured mask mounting system. It depicts the mask housing, the overall external view of the filtration system, the front pre-filter system, the contoured mask mounting system with the elastic face-contoured seal and moisture absorbing liner, the mounting strap and the under-ear strap.

FIG. 3 is the sectioned illustration of the multi-stages electronic filtration with potential feedback system, which depicts a portion of the direct air purge blockage cover, pre-filter stage filter element, a portion of the mask, a portion of the filter housing, a portion of the ionic filtration with potential feedback stage filter element, a portion of the electrostatic filtration stage filter element, a portion of the UV ray generating device element, a portion of the UV ray leakage protection system, a portion of the moisture collection system, a portion of the electrical connection from the cable to the ionizing pins subassembly, the distance between the front pre-filter system, collector module and the ion generating system, a portion of the electrical connection from the cable to the electrostatic filter subassembly according to present invention.

FIG. 4 is the illustration showing the sectioned view as per FIG. 3 with negative ions released by the pointed sharp ends of the ionic filtration chamber and the electrostatic charges established in the electrostatic filtration chamber, which depicts portion of the distance between the front pre-filter stage filter element, a portion of the collector module and a portion of the ion generating system that contribute to the electromechanical potential feedback functions.

FIG. 5 illustrates the sectioned electronic multi-stages filtration mechanism system, it depicts a portion of the UV ray generating system, a portion of the UV ray reflection system inside the mask chamber, a portion of the collector module and a portion of the UV ray leakage protection system.

FIG. 6 illustrates the electronic air filter element with potential feedback mechanism system during the inhalation operation of the user, which depicts a portion of the multi-stage electronic filtration element, a portion of the mask housing, a portion of the user face, a portion of the air-flow during inhalation by user.

FIG. 7 illustrates the electronic air filter element with potential feedback mechanism system during the exhalation operation of the user, which depicts a portion of the multi-stage electronic filtration element, a portion of the mask housing, a portion of the user face, a portion of the air-flow during exhalation by user.

FIG. 8 illustrates the mechanism of the electro-mechanical potential feedback system inside the mask chamber. which depicts a portion of the multi-stage electronic filtration element, a portion of the electronic control device, a portion of the multi-meter for electrical current measurement, a portion of the power supply and a portion of the connection cable connecting all the device together electrically.

FIG. 9 illustrates the application of the present invention when being adapted to eye protection goggles to form a face-mask with Potential Feedback Human Breath Filtration Device, which depicts a portion of the user head and body, a portion of the eye protection goggles, a portion of the Potential Feedback Human Breath Filtration Device, and a portion of the electronic control system.

FIG. 10 illustrates the application of the present invention when being adapted to a user head covering hood with eye protection to form a hood-mask with Potential Feedback Human Breath Filtration Device, which depicts a portion of the user head and body, a portion of the hood with eye protection, a portion of the Potential Feedback Human Breath Filtration Device.

FIG. 11 illustrates the application of the present invention when being adapted to a helmet to form a helmet-mask with Potential Feedback Human Breath Filtration Device, which depicts a portion of the user head and body, a portion of the helmet, a portion of the Potential Feedback Human Breath Filtration Device.

FIG. 12 illustrates the ionic flux sensing device being used to sense the present of ions being generated by the ionization system by capturing the ions at its sensing antenna.

FIG. 13 illustrates the ionic flux sensing device being used with its sensing antenna to sense the present of electrical flux energy being generated by the positive collector receiving the ions generated by the ionization system.

FIG. 14 illustrates the ionic flux sensing device being used with its sensing antenna to sense the present of electrical flux energy being generated by the Potential Feedback Ionization Human Breath Filtration Device system.

### DETAILED DESCRIPTION OF THE PREFERRED EMBODIMENT

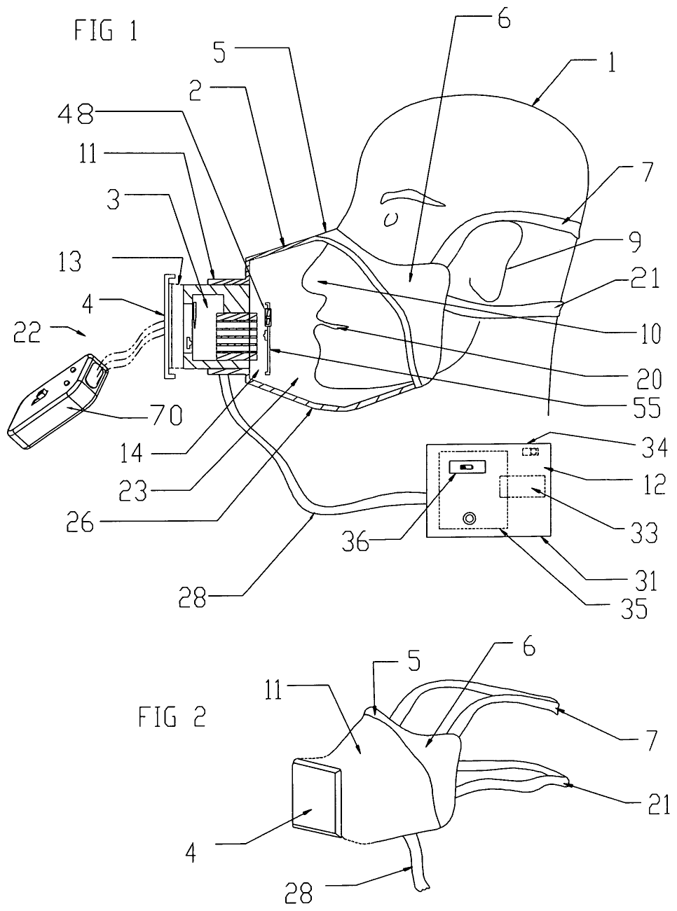

FIG. 1 is the overall system of the Potential Feedback Ionization Human Breath Filtration Device system 2. The overall system 2 is comprised of 4 subsystems namely the filtration system 11, the control system 12, the contoured mask mounting system 6 and the ionic flux sensing device 70.

This is a generic system that can be operated as respirator mask, adapted to goggles to form face-mask, adapted to a head covering hood to form a hood-mask and adapted to a helmet to form a helmet with mask to provide air purification with said devices.

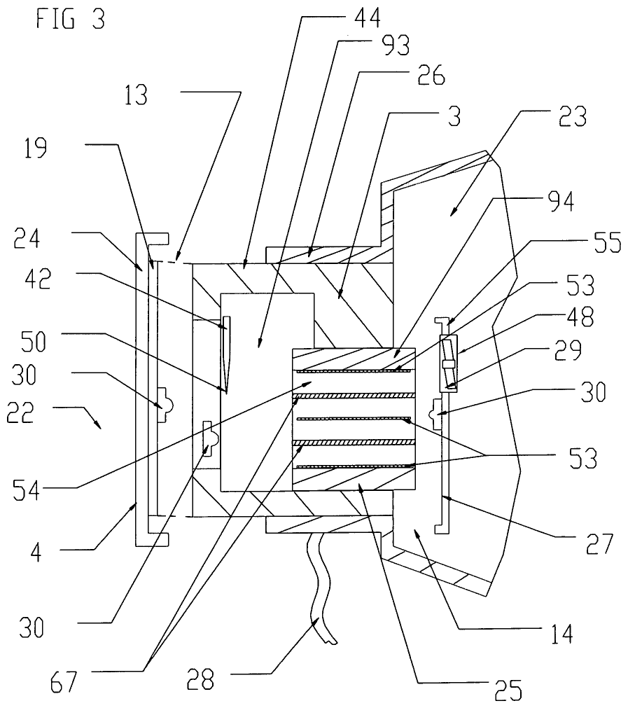

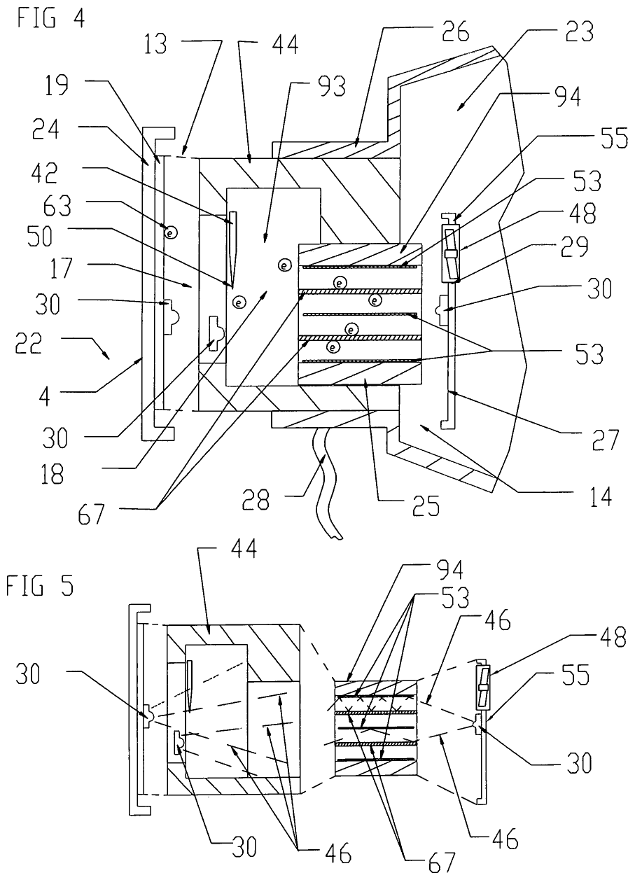

The filtration system 11 includes the mask housing 26, an ionization with potential feedback filter element module 3, a front static pre-filter cover system 4 and a rear anti-UV ray leakage cover system 55. This filtration system 11 is shown in cross section view and is further detailed in FIG. 3. The front static pre-filter cover system 4 is mounted to the outside of the mask housing 26. The assembly can be by snap on, or by fastener which can facilitate the assembly means. The front static pre-filter cover system 4 provides blockage to direct air purge due to wind blowing directly into the mask housing 26 with a protective cover while providing a sufficient air passage for the air to pass from the ambient 22 along the front edge opening 13 to the ionization with potential feedback filter element module 3 with very low resistance at low flow rate as human inhaling breaths breathing air flow rate. It also provides a sufficient air passage for the air to pass to the ambient 22 from the ionization with potential feedback filter element module 3 with very low resistance at low flow rate as user exhaling. The ionization with potential feedback filter element module 3 is mounted inside the center opening of the mask housing 26. The assembly can be by snap on, press-fitting, or by fastener which can facilitate the assembly means. This ionization with potential feedback filter element module 3 will filter and capture the particles entering inside the module carried by air stream induced by breath of the user 1. The rear anti-U V ray leakage cover system 55 is mounted to the outside of the rear side of the mask housing 26 next to the ionization with potential feedback filter element module 3. The assembly can be by snap on or by fastener which can facilitate the assembly means. The rear anti-UV ray leakage cover system 55 provides protective cover with a sufficient air passage at the rear edge opening 14 for the air to pass from the mask chamber 23 to the ionization with potential feedback filter element module 3 with very low resistance at low flow rate as human exhaling breath. It also provides a positive air pressure for the air to pass to the mask chamber 23 from the ionization with potential feedback filter element module 3 with positive air flow pressure to ease human inhaling breath. The rear anti-UV ray leakage cover system 55 also blocks off contaminants from sneeze and saliva of the user 1 from entering the ionization with potential feedback filter element module 3. The rear anti-UV ray leakage cover system 55 is also equipped with a brushless DC motor driven impeller device 48. This brushless DC motor driven impeller device 48 provides the sufficient positive air pressure to balance the originally very low air flow resistance of the system and additional air pressure to help the user to breathe more comfortable.

The mask housing 26 provides a rigid contoured shape covering the nose 10 and mouth 20 of the user 1; and a mounting support to accommodate the front static pre-filter cover system 4, the multi-stages filter element module 3 and the rear anti-UV ray leakage cover system 55. The mask housing 26, front static pre-filter cover system 4 and the rear anti-UV ray leakage cover system 55 can be made of metal, plastic, paper product, fiberglass or carbon fiber material. The best choice and most cost-effective method of producing this mask housing 26 is by plastic molding to achieve the shape and rigidity supporting the function of the mask housing 26.

The contoured mask mounting system 6 is consisted of an elastic face-contoured seal 5, a moisture collection system 8, a mounting strap 7, a under ear strap 21 on each ear of the user 1. The elastic face-contoured seal 5 is assembled to the mask housing 26 by snap on, press-fitting, or by fastener which can facilitate the assembly means. It is made of elastic material such as rubber, silicon rubber, foam pad, nylon or any other material which can facilitate a soft, flexible sealing function of the contoured seal 5. It can be made of one single piece part or an assembled piece part to facilitate the functions of the contoured seal 5. The moisture collection system 8 attached to the edges of the contoured seal 5 to provide soft and gentle contact surfaces to the user's face and is further descripted in FIG. 3. It is made of medically treated material to protect user from allergic reaction. It is also made of moisture absorption material to collect any moisture formed inside the mask due to condensation of high moisture content in exhaling air by the ionization processes.

The mounting strap 7 is with both ends assembled to the contoured seal 5 or the mask housing 26. The mounting strap 7 is to be worn the way that it rests on the ears 9 of the user 1 and wraps around the back of the head of user 1. The under-ear strap 21 is with one end assembled to the contoured seal 5 or the mask housing 26, and the other end assembled to the mounting strap 7 surrounding the ear of the user 1. In result, the filtration system 11 is firmly mounted to cover the mouth and nose of the user 1 with the contoured seal 5 resting on the nose and cheek of the user 1. The elastic contoured seal 5 separates the mask chamber 23 from the ambient 22 by forming a seal along the contour of the face and chin of the user 1. The ionization with potential feedback filter element module 3 becomes the only air passage between the air in the mask chamber 23 and the ambient 22. The driving mechanism for the air exchange is the breathing process of user 1 with additional help of increased air pressure of a brushless DC motor driven impeller device 48 with air movement from ambient 22 to mask chamber 23 caused by inhalation and air movement from mask chamber 23 to ambient 22 caused by exhalation of user 1.

The control system 12 consists of a control unit 31 which is equipped with the main PCBA (printed circuit board assembly) 35 with connection to the battery 33 and a power selector on/off switch 34. The main PCBA (printed circuit board assembly) 35 is equipped with electronic components and with the power selector on/off switch 34 set at “ON” position; the main PCBA (printed circuit board assembly) 35 will generate high negative voltage functions to activate the ionization with potential feedback filter element module 3 via the connector cable 28. The connector cable 28 connects the mask housing 26 and the control unit 31. The control unit 31 is also equipped with a status indicator 36 showing the ON/OFF status of the system. The control unit 31 can receive power source input from an USB power outlet via the USB power conversion cable 43.

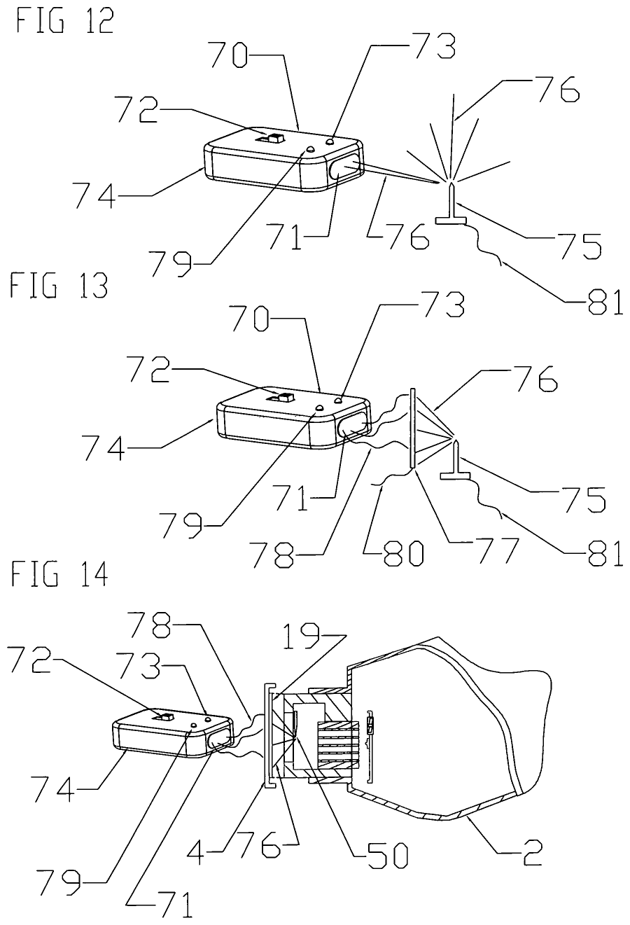

The ionic flux sensing device 70 is provide to the user such that the user can easily verify the overall system 2 is functioning. The functionality and usage of the ionic flux sensing device 70 is further descripted in FIG. 12, FIG. 13 and FIG. 14.

FIG. 2 is the front isometric view of the filtration system 11 with the contoured mask mounting system 6. The front static pre-filter cover system 4 is assembled to mask housing 26 covering the front air entrance of the filtration system 11. The mounting strap 7 is supported by the elastic face-contoured seal 5 and/or the mask housing 26 at both ends. The under-ear strap 21 is supported by the elastic face-contoured seal 5 and/or the mask housing 26 at one end and attached to the mounting strap 7 at the upper end. The connection cable 28 is connected to the mask housing 26. The mounting strap 7 may be made of rubber, silicon rubber, nylon, nylon base cloth like material, cotton base cloth like material; and may be made up of more than one single part for easier mounting and dismounting.

FIG. 3 is the section view of the filtration system 11 with the basic structural support of the mask housing 26. The rear anti-UV ray leakage cover system 55 is placed at the inside entrance of the ionization with potential feedback filter element module 3. This rear anti-UV ray leakage cover system 55 consists of a rear cover 29, an electric brushless DC motor driven impeller device 48 and a Light Emitting Diode with wavelength shorter than 440 Å 30 which is electrically connected through the mask housing 26 via the connector cable 28 to the control unit 31. All the electrical connections of the electrical components also including the brushless DC motor driven impeller device 48, the Light Emitting Diode with wavelength shorter than 440 Å 30 are covered by a layer of transparent non-conductive plastic coating for electrical shortage protection. The inner surface 27 of the rear cover 29 is non-reflective and has a light absorption material coating so that the UV rays from the ionization with potential feedback filter element module 3 will not leak through the rear edge opening 14 into the inner mask chamber 23. The light ray emitting direction of the Light Emitting Diode with wavelength shorter than 440 Å 30 is towards the electrostatic filtration system 94.

The moisture collection system 8 is comprised of mask edge liner 69, which is made of soft material with moisture absorption feature. Mask edge liner 69 wraps along the edges especially the inside portion of the elastic face-contoured seal 5 providing soft and gentle contact surfaces to the user's face. Moisture from user's exhalation breath will condense inside the mask by the ionization process. After extended time moisture collected become water drops. The mask edge liner 69 will absorb these water drops keeping the water drops from contacting the user's face.

The ionization with potential feedback filter element module 3 is in the middle of the mask housing 26. It is assembled to the mask housing 26. The assembly can be by snap on, press-fitting, or by fastener which can facilitate the assembly means. The ionization with potential feedback filter element module 3 is comprised of two filtration system namely the ionic filtration system 93 and the electrostatic filtration system 94 enclosed in the filter housing 44. The ionic filtration system 93 consists of a highly charged negative (−) electrode 42 with sharp metallic needle points 50 connected to it and are locating in the center portion of the ionic filtration system 93. The negatively (−) charged electrode 42 is assembled to the filter housing 44.

The electrostatic filtration system 94 consists of collector module housing 25, parallel sets of negatively charged electrode fins 53 sandwiching with positively charged electrode fins 67. An electrostatic field is formed between a negatively charged electrode fin 53 and positively charged electrode fin 67. The strength of the electrostatic field is determined by the gap width A 54 between the two oppositely charged electrodes and the potential difference between them. Further detail explanation of the filtration processes is illustrated in FIG. 4. The negatively charged electrode fins 53 are mounted inside the collector module housing 25. The positively charged electrode fins 67 are mounted inside the collector module housing 25 but insulated from positively charged electrode fins 67. Both positively charged electrode fins 67 and negatively charged electrode fins 53 are electrically connected to the control unit 31 via the mask housing 26 and connector cable 28.

The front static pre-filter cover system 4 is placed at the outside entrance of the ionization with potential feedback filter element module 3. It is comprised of front shield 24, dust collector 19 and Light Emitting Diode with wavelength shorter than 440 Å 30. The Light Emitting Diode with wavelength shorter than 440 Å 30 and the dust collector 19 are electrically connected through the mask housing 26 via the connector cable 28 to the control unit 31. The light ray emitting direction of the Light Emitting Diode with wavelength shorter than 440 Å 30 is towards the electrostatic filtration system 94. The surfaces of the dust collector 19 reflect UV light rays from the ionic filtration system 93 back towards the electrostatic filtration system 94.

This front static pre-filter cover system 4 is assembled to the mask housing 26. The assembly can be done by snap on, press-fitting, or by fastener which can facilitate the assembly means.

FIG. 4 is the section view of the filtration system 11 illustrating the ionization status of the ionic filtration system 93 and the electrostatic charged status of the electrostatic filtration system 94. Within the ionic filtration system 93, needlepoint 50 produces high levels of negative ions 63 when high negative DC voltage is applied to it. This is the by far most effective way of negative ions 63 generation and will help to clean the air inside the ionic chamber 64. The negative ions 63 generated will cause electrons to adhere to molecules of Oxygen, Nitrogen and other trace gases in the inhaling or exhaling air from the user 1's breath. This process creates ions with a negative charge as negative ions 63. When the negative ions 63 will collide with airborne pollutants such as pollen, mold spores, dust, bacteria, tobacco smoke, saliva moisture, sneeze moisture and many other airborne particles. The negative charge of negative ions 63 is then transferred to the airborne particles. Surrounding this newly negatively charged particle are many other particles that are positively charged. These positively charged particles are drawn to the negatively charged particle and begin to build-up, eventually these particles become too heavy and fall harmlessly off from the air stream. The other negatively charged airborne particles will then be attracted to the positively charged collector conductors, the positively charged electrode fin 67, when traveling along the air stream.

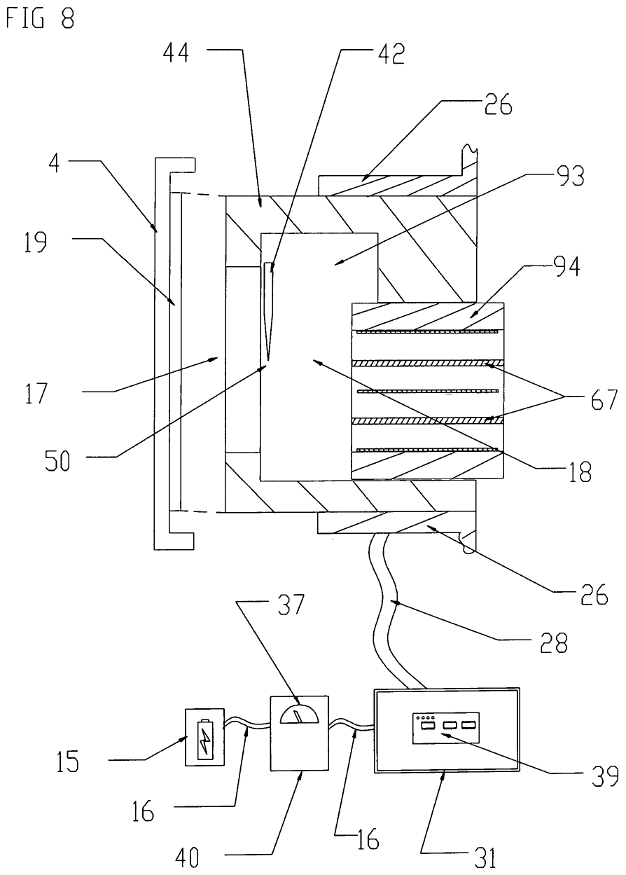

The pre-filter air gap 17 is the distance between the needle point 50 and the closest spot on the surface of the positively charged dust collector 19. When this pre-filter air gap 17 getting smaller, the rate of ionization increases and so as the power consumption of the main control unit 31 increases accordingly. The increase in the rate of ionization is materializing in the output of ions 63 generated inside the ionic filtration system 93. This addition of ions 63 increases cleaning power of the ionic filtration system 93. This is the potential feedback ionization process and it represents at least 5% increases on the power consumption of the main control unit 31 due to the pre-filter air gap 17 getting smaller as compare to when the pre-filter air gap 17 is over 30 mm apart. The pre-filter air gap 17 should be limited to before lightning effect occurs when the needle point 50 and the positively charged dust collector 19 getting too close to each other, the ions generated concentrate together and form a lightning strike towards the positively charged dust collector 19. The control of pre-filter air gap 17 is further descripted in FIG. 8.

In the electrostatic filtration system 94, a high negative voltage is induced to the negatively charged electrode fin 53 and the positively charged electrode fin 67 is connected to the electrically positive. It results that the surface of the negatively charged electrode fin 53 will be highly negatively charged 66 and the causing an electrostatic field to form between the negatively charged electrode fin 53 and the positively charged electrode fin 67, which becomes equally highly positively charged 65. This electrostatic field is an uniform electric field of force and causes an uniform distribution of electrons (negative charge 66) on the surface of negatively charged electrode fin 53, and an equal and uniformly distributed deficiency of electrons (positive charge 65) on the positive fin 67. The voltage graduation is uniform throughout this field, except at its edges and near sharp corners of the plates/fins.

The collector module air gap 18 is the distance between the needle point 50 and the closest spot on the surface of the positively charged electrode fins 67. When this pre-filter air gap getting smaller, the rate of ionization increases and so as the power consumption of the main control unit 31 increases accordingly. The increase in the rate of ionization is materializing in the output of ions 63 generated inside the ionic filtration system 93. This addition of ions 63 increases cleaning power of the ionic filtration system 93. This is the potential feedback ionization process and it represents at least 5% increases on the power consumption of the main control unit 31 due to the pre-filter air gap 17 getting smaller as compare to when the pre-filter air gap 17 is over 30 mm apart. The pre-filter air gap 17 should be limited to before lightning effect occurs when the needle point 50 and the positively charged electrode fins 67 getting too close to each other, the ions generated concentrate together and form a lightning strike towards the positively charged electrode fins 67.

A single airborne particle charged by the potential feedback ionization process becomes highly negatively charged particle before entering electrostatic filtration system 94 and is acted upon by a force equaling the sum of all attracting and repelling forces. The negatively charged particles will be attracted and stick to the positively charged surfaces of the positively charged electrode fins 67.

The particles that are collected and are in physical contact with the positively charged collector fins 67 lose their “opposite charge” and take on the charge of the respective collector fins. They remain attached to the collector fins because of molecular adhesion and due to cohesion to other particles already collected. As a result, contaminants are removed from the air stream of breath induced by the user 1's inhalation and exhalation efforts. In practice, the filtration system 11 will charge floating particles as small as 0.01 micron and drive them to adhere to the collector plates where they will stay attached.

FIG. 5 is the section view of portion of the filtration system 11 where the UV light rays 46 generated by the Light Emitting Diode with wavelength shorter than 440 Å 30 will shine onto the positively charged surfaces of the electrode fins 67. The surfaces of negative fins 53 and the positively charged electrode fins 67 are reflective surfaces. The UV light rays 46 generated by Light Emitting Diode with wavelength shorter than 440 Å 30 will be reflecting back and forth inside the ionization filtration system 93 and electrostatic filtration system 94; eventually all the substances attaching to the positively charged surfaces of the electrode fins 67 are under UV light rays 46 exposure.

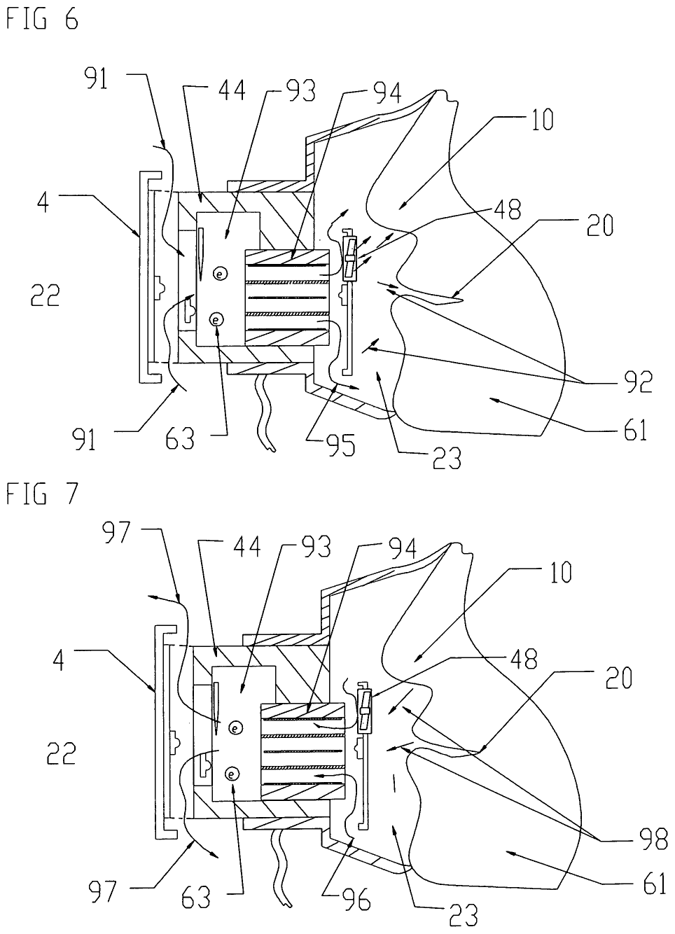

FIG. 6 is the section view of the filtration system 11 with the user 1 inhaling through the filtration system 11. The inhaling breath of user 1 and brushless DC motor driven impeller device 48 become the power source to draw the air stream A 92 from the mask chamber 23 into the user 1's nose 10 and mouth 20. As results, the air pressure in the mask chamber 23 will be lower than the air pressure in the electrostatic filtration system 94 and cause the air stream B 95 in the electrostatic filtration system 94 to flow through the rear anti-UV ray leakage cover system 55 into the mask chamber 23. In the same token the air in the ionic filtration system 93 will flow into electrostatic filtration system 94; and the air stream C 91 in the ambient 22 will flow through the front static pre-filter cover system 4 to the ionic filtration system 93. Eventually, during the inhalation process, the air flow from the ambient 22 through front static pre-filter cover system 4, the ionic filtration system 93, the electrostatic filtration system 94 and the rear anti-UV ray leakage cover system 55 into user 1's nose 10 and mouth 20. When the desirable voltage potential is applied to the filtration system 11, the ionic filtration system 93 and the electrostatic filtration system 94 will remove most of the air borne particles, contaminants and substances from the inhaling air stream and supplying only very clean air to the user 1. During the filtration processes, the air stream is free to move from one stage to the other and there will be no resistance induced to the inhalation effort. This is an advantage of this invention over the conventional filtration by filter material type nose mask. Weaker users 1 especially those with breathing difficulty like Asthma will find this electronic inhalation and exhalation breath filtration device system 2 very comfortable to use.

FIG. 7 is the section view of the filtration system 11 with the user 1 exhaling through the filtration system 11. The exhaling breathe becomes the power source to drive the air stream D 98 from the user 1's nose 10 and mouth 20 to the mask chamber 23. As results, the air pressure in the mask chamber 23 will be higher than the air pressure in the electrostatic filtration system 94 and cause the air stream E 96 in the mask chamber 23 to flow through the rear anti-UV leakage cover system 55 into the electrostatic filtration system 94. In the same token the air in the electrostatic filtration system 94 will flow to the ionic filtration system 93; and the air stream G 97 in the ionic filtration system 93 will flow through the front static pre-filter cover system 4 to the ambient 22. Eventually, during the exhalation process, the air flow from the user 1's nose 10 and mouth 20 through rear anti-UV ray leakage cover system 55, the electrostatic filtration system 94, the ionic filtration system 93 and the front static pre-filter cover system 4 into ambient 22. When the desirable voltage potential is applied to the filtration system 11, the ionic filtration system 93 and the electrostatic filtration system 94 will remove most of the air borne particles, contaminants and bacteria from the exhaling air stream and supplying only very clean air to the ambient 22. During the filtration processes, the air stream is free to move from one stage to the other and there will be no resistance induced to the exhalation effort. This is an advantage of this invention over the conventional filtration by filter material type nose mask. The exhaling air will pass through the filtration system 11 and be filtered rather than leaking through the edges as of using paper filter nose mask where the exhaling air finds easier way out. The front static pre-filter cover system 4, the electrostatic filtration system 94 can be removed from the system to be cleaned separately.

FIG. 8 illustrates the advantage of the electro-mechanical potential feedback technology and is showing the section view of the filtration system 11 connecting to the control unit 31 via the connector cable 28. The control unit 31 is receiving the electrical power from constant voltage power source 15 through connection cable 16, electricity current meter 40 and another connector cable 16.

When filtration system 11 is in operation, Electrical power from constant voltage power source 15 is supplied to the control unit 31 and the electronic control circuit 39 inside the control unit 31 will generate a high negative voltage and send to the negative electrode 42 of the filtration system 11. Then ionization occurs at the sharp metallic needle point 50 and streams of ion air stream 76 are released in all directions. These ion air streams 76 will eventually bombard onto the surfaces of the positively charged metallic dust collector 19 and the positively charged electrode fins 67. The prefilter air gap 17 is the distance between the sharp metallic needle point 50 and the positively charged metallic dust collector 19. The collect module air gap 18 is the distance between the sharp metallic needle point 50 and the positively charged electrode fin 67.

The prefilter air gap 17 and the collect module air gap 18 play a very important role in the filtration system 11. When the prefilter air gap 17 becomes very small like 1 mm distance, lightning occurs due to streams of ion air streams 76 just leaving the sharp metallic needle point 50 bombard directly through the prefilter air gap 17 on to the positively charged metallic dust collector 19. In this phenomenon there will not be ionization air filtration occur because all the electrical energy strike onto the positively charged metallic dust collector 19 due to the high positive and negative electrical potential difference. As the prefilter air gap 17 becomes wider lightning phenomenon will not happen and ionization air filtration occurs. The electrical current drawing through the control unit 31 is shown on the electricity current meter 40 with X value 37. The X value 37 will keep dropping as the air gap 17 becomes larger and larger. When the prefilter air gap 17 becomes over 30 mm the rate of change of X value 37 will become very small and not noticeable. The best X value 37 is to keep the prefilter air gap 17 at between 3 mm to 8 mm. This will increase the X value 37 to its optimal level. It will optimize the amount of ionization producing more ions in the air to perform the ionization air purification operation while at a lower voltage level. This will in turn minimize the amount of Ozone produced during ionization process. It is because rate of Ozone produced is directly proportional to the increase of negatively charging voltage at the negative electrode 42 and the ions releasing from the sharp metallic needle point 50.

Without the present of the positively charged metallic dust collector 19 close by the sharp metallic needle point 50, ions generated from the sharp metallic needle point 50 are scatter and shoot off randomly. Now with the present of the positively charged metallic dust collector 19 close to the sharp metallic needle point 50 with an effective prefilter air gap 17 at between 3 mm to 8 mm distance; the ions generated are concentrated with the ion air streams 76 flowing from the sharp metallic needle point 50 towards the positively charged metallic dust collector 19. It results that the prefilter air gap 17 has much higher ion concentration than the rest of the space around and increases the rate of ionic air cleaning process when air borne particles are passing through.

Similarly, when the collector module air gap 18 becomes very small like 1 mm distance, lightning occurs due to streams of ion air streams 76 just leaving the sharp metallic needle point 50 bombard directly through the collector module air gap 18 on to the positively charged electrode fin 67. In this phenomenon there will not be ionization air filtration occur because all the electrical energy strike onto the positively charged electrode fin 67 due to the high positive and negative electrical potential difference. As the collector module air gap 18 becomes wider lightning phenomenon will not happen and ionization occurs. The electrical current drawing through the control unit 31 is shown on the electricity current meter 40 with X value 37. The X value 37 will keep dropping as the collector module air gap 18 becomes larger and larger. When the collector module air gap 18 becomes over 30 mm the rate of change of X value 37 will become very small and not noticeable. The best X value 37 is to keep the collector module air gap 18 at between 4 mm to 8 mm. This will increase the X value 37 to its optimal level. It will optimize the amount of ionization producing more ions in the air to perform the ionization air purification operation while at a lower voltage level. This will in turn minimize the amount of Ozone produced during ionization process. It is because rate of Ozone produced is directly proportional to the increase of negatively charging voltage at the negative electrode 42 and the ions releasing from the sharp metallic needle point 50.

Without the present of the positively charged electrode fin 67 close by the sharp metallic needle point 50, ions generated from the sharp metallic needle point 50 are scatter and shoot off randomly. Now with the present of the positively charged electrode fin 67 close to the sharp metallic needle point 50 with an effective collector module air gap 18 at between 3 mm to 8 mm distance; the ions generated are concentrated with the ion air streams 76 flowing from the sharp metallic needle point 50 towards the positively charged electrode fin 67. It results that the collector module air gap 18 has much higher ion concentration than the rest of the space around and increases the rate of ionic air cleaning process when air borne particles are passing through.

The electro-mechanical potential feedback technology is defined as the control of the prefilter air gap 17 and the collector module air gap 18 such that it can maximize the rate of air purification results with lower voltage at the negative electrode 42 such that minimum amount of Ozone will be generated during the operation. The optimal increase of electrical current shown on X value at the electricity current meter 40 will be at least +5% from its lowest at Y value 61 which is the minimum current reading when ionization just begins to occur at the sharp metallic needle point 50.

FIG. 9 is the front view illustrating the application of the Potential Feedback Ionization Human Breath Filtration Device system 2 being adapted to incorporated together with a goggle 99 to form a face mask with respirator system 90. The face mask with respirator system 90 is to be adapted to the face of user 1. The air inside the mask chamber 23 is free to flow to the chamber covered by the eye goggle 99 resulting that the air surrounds the user 1's eye is also cleaned by the Potential Feedback Ionization Human Breath Filtration Device system 2.

FIG. 10 is the front view illustrating the application of the Potential Feedback Ionization Human Breath Filtration Device system 2 being adapted to incorporated together with a head covering hood 59 with built in lens 58 to cover and protect the eyes and the head of the user 1. The air inside the mask chamber 23 is free to flow to the chamber covered by the lens 58 and the hood 59, resulting that the air surrounds the user 1's eye and head is also cleaned by the Potential Feedback Ionization Human Breath Filtration Device system 2.

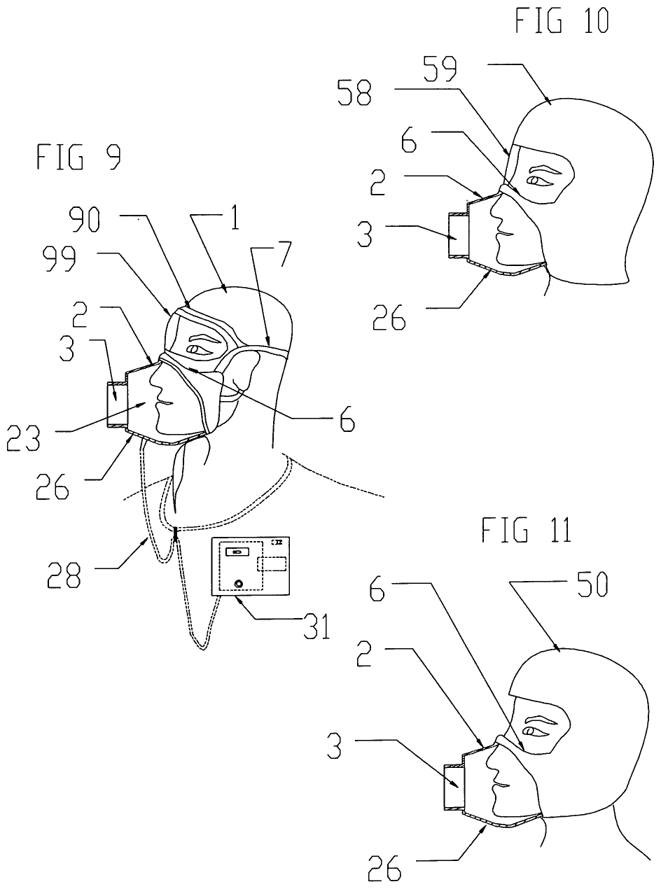

FIG. 11 is the front view illustrating the application of the Potential Feedback Ionization Human Breath Filtration Device system 2 being adapted to incorporated together with a head covering helmet 51 protecting the head of the user 1. Cleaned is provided to the user by the Potential Feedback Ionization Human Breath Filtration Device system 2.

FIG. 12 is the ionization flux sensing device 70 in operation on sensing the presence of ionization. The ionization flux sensing device 70 is comprised of a sensing antenna 71, sensor power switch 72, sensor power LED 73, sensor electronic unit 74 and sensor flux strength LED 79.

With the ionization flux sensing device 70 in operation, the sensor power switch 72 is at power on position and the sensor power LED 73 lights up. High negative voltage is sent to the ion generating pin 75 by the wire 81 and ionization occurs. Ionic air stream 76 carry the negative ions leaving the tip of the ion generating pin 75 in all directions. Some of these ionic air streams 76 bombard onto the surface of the sensing antenna 71. These collected ionic air streams 76 will transform into a small electrical current and is sent to the sensor electronic unit 74 of the ionization flux sensing device 70, The small current will be magnified to over 50,000 time by the sensor electronic unit 74 to be able to light up the sensor flux strength LED 79. Depending on initial amount of ionic air stream 76 received, the sensor flux strength LED 79 will light up from not lighting up at all to dim or to very bright. The intensity of the sensor flux strength LED 79 from none to very bright provides user an obvious visual observation to identify the present of ionization.

FIG. 13 is the ionization flux sensing device 70 in operation on sensing the presence of electrical flux field 78. The ionization flux sensing device 70 is comprised of a sensing antenna 71, sensor power switch 72, sensor power LED 73, sensor electronic unit 74 and sensor flux strength LED 79.

With the ionization flux sensing device 70 in operation, the sensor power switch 72 is at power on position and the sensor power LED 73 lights up. High negative voltage is sent to the ion generating pin 75 by the wire 81 and ionization occurs. Ionic air streams 76 carry the negative ions leaving the tip of the ion generating pin 75. A positively charged metal plate 77 is connected to electrically positive charge via the wire 80 and is placed close to the ion generating pin 75. Due to the electrical potential difference the ionic air streams 76 leaving the tip of the ion generating pin 75 are attracted to flow towards the positively charged metal plate 77. These ionic air streams 76 bombard onto the surface of the positively charged metal plate 77. As results an electrical flux field 78 is formed on the opposite surface of the positively charged metal plate 77. This electrical flux field 78 will act on the surface of the sensing antenna 71. These collected electrical flux field 78 will transform into a small electrical current and is sent to the sensor electronic unit 74 of the ionic flux sensing device 70, The small current will be magnified to over 50,000 time by the sensor electronic unit 74 to be able to light up the sensor flux strength LED 79. Depending on initial amount of electrical flux field 78 received, the sensor flux strength LED 79 will light up from not lighting up at all to dim or to very bright. The intensity of the sensor flux strength LED 79 from none to very bright provides user an obvious visual observation to identify the present of ionization.

FIG. 14 is the ionization flux sensing device 70 in operation to check if ionization occurs against the Potential Feedback Ionization Human Breath Filtration Device system 2.

With the ionic flux sensing device 70 in operation, the sensor power switch 72 is at power on position and the sensor power LED 73 lights up. High negative voltage is sent to the ion generating pin 50 of the Potential Feedback Ionization Human Breath Filtration Device system 2 and ionization occurs. Ionic air streams 76 carry the negative ions leaving the tip of the ion generating pin 50. The metallic dust collector 19 of front static pre-filter cover system 4 is electrically positively charged. Due to the electrical potential difference between the ion generating pin 50 and the metallic dust collector 19, the ionic air streams 76 leaving the tip of the ion generating pin 50 are attracted to flow towards the positively charged metallic dust collector 19. These ionic air streams 76 bombard onto the surface of the positively charged metallic dust collector 19. As results an electrical flux field 78 is formed on the opposite surface of the positively charged metallic dust collector 19. This electrical flux field 78 will act on the surface of the sensing antenna 71. These collected electrical flux field 78 will transform into a small electrical current and is sent to the sensor electronic unit 74 of the ionization flux sensing device 70, The small current will be magnified to over 40,000 time by the sensor electronic unit 74 to be able to light up the sensor flux strength LED 79. Depending on initial amount of electrical flux field 78 received, the sensor flux strength LED 79 will light up from not lighting up at all to dim or to very bright. The intensity of the sensor flux strength LED 79 from none to very bright provides user an obvious visual observation to identify the present of ionization of the Potential Feedback Ionization Human Breath Filtration Device system 2.

It will be appreciated that the sizes, quantities, shapes and dispositions of various components like needlepoint ionization pins, electrode fins, electro-collectors, louver covers, conductor leads, wires, cable length, material use, filter size, filter gap clearance, size of the mask and size of the seal can be varied, without departing from the spirit and scope of the invention. Similarly, the sizes and contour of the nose mask, face mask and hood with reference to adult, children, male and female, and the like may be varied. While the methods of connecting the service loop of the cable are illustrated, other methods may instead be used to facilitate the concept of service loop. While the methods of mounting the mask-filter system with straps concept is illustrated, other methods may instead be used to facilitate the concept of mounting to the user's face. While this Potential Feedback Human Breath Filtration Device system has been described with respect to application to nose mask, face mask, helmet and hood, the described system may also apply to other human wearing electronic filtration systems and may have more than one air inlet or air outlet.

Modifications and variations may be made to the disclosed embodiments without departing from the subject and spirit of the invention as defined by the following claims.

What is claimed is:

1. A portable potential feedback human breath filtration device adapted to be carried on a human body, comprising a filtration apparatus which is adapted to interact with human inhalation and exhalation efforts with help of an electric fan as energy source to drive the breathing air of the user through the said filtration apparatus to remove airborne matters in the air and is further comprised of
a mask body including a mask cavity adapted to surround the nose and mouth of a user and a filter cavity for an electronic air filter element with potential feedback function to be installed;
a seal, which is supported by the mask body, separates the mask chamber from the ambient by forming a sealing edge along the edge of the mask module and the skin surface of the user when the said device is being adapted to be worn onto the face by the user;
a mounting strap, which is supported by the mask module is adapted to be wrapped above the ears and around the back of the head of the user to keep the mask module covering the nose and mouth of the user;
a layer of transparent non-conductive conformal coating covering all the electrical joints to prevent from electrical shortage;
a water condensation collection system to absorb the water condensation from the moisture within the breaths of the user during the ionization operation;
an electronic air filter element with potential feedback function is installed inside the filter cavity of the mask body;
an electric fan;
a front static pre-filter cover system covers the front entrance of the electronic air filter element with potential feedback function is mounted to the front side of the mask module;
a rear UV generating device with anti-UV leakage cover system covers the back entrance of the electronic filter element is mounted to the inside of the mask module;
a control system consists of printed circuit board assembly, electronic components and battery provides electronic functions to operate the said electronic filter element;
a connection cable connects the said electronic filter element to the said control system;
an USB power conversion cable;
an ionization flux sensing device.

2. The apparatus of claim 1, wherein said control system of a portable potential feedback human breath filtration device adapted to be carried on a human body comprises
a main printed circuit board assembly with electronic components, which also includes a high voltage power supply generating electronic components;
a battery connected to the said main printed circuit board assembly to supply power to the said control system;
a USB power conversion cable;
a selection switch to allow the said control system of a portable potential feedback human breath filtration device to select the power source from said internally installed battery and external electrical power source;
a status indicator connected to the said main printed circuit board assembly to show the charge level of the battery, the power level setting selected and if the power is on;
an enclosure housing for all the above components to be mounted to.

3. The apparatus of claim 1, wherein said electronic air filter element with potential feedback function is a multiple stages electronic filtration element which comprises
a direct air purge blockage cover
a pre-filter stage filter element;
an ionic filtration with potential feedback stage filter element;
an electrostatic filtration stage filter element;
an ultraviolet ray (UV) generating device element;
an ultraviolet ray (UV) reflection system;
an ultraviolet ray (UV) leakage protection system;
a rear anti-UV leakage cover system;
an electric fan.

4. The apparatus of claim 3, wherein said ionic filtration with potential feedback stage filter element comprises
a conducting collector element;
an electrical coupling means for receiving an electrical potential from a high voltage source;
an ionizing element comprising an electrically conductive material having pointed sharp ends for providing a high potential gradient to ionize particle components of a gas passing there-through, said conducting collector element and ionizing element being connected to the electrical coupling means to produce said high potential gradient when supplied with charge from a high voltage source through said electrical coupling means;
the distance between the said electrically conductive material having pointed sharp ends measuring from the tips of the sharp ends and the positively charged surface of the said pre-filter stage filter element is close enough to affect the increase of electrical power consumption of electrically operating the control unit by at least 5% as compare to when the said distance of the said electrically conductive material having pointed sharp ends and the said positively charged surface of the said pre-filter stage filter element is at 30 mm apart at the closest location measuring from the tips of the sharp ends;
the distance between the said electrically conductive material having pointed sharp ends measuring from the tips of the sharp ends and the positively charged surface of the electrostatic filtration stage filter element is close enough to affect the increase of electrical power consumption of electrically operating the control unit by at least 5% as compare to when the said distance of the said electrically conductive material having pointed sharp ends and the said positively charged surface of the said pre-filter stage filter element is at 30 mm apart at the closest location measuring from the tips of the sharp ends.

5. The apparatus of claim 4, wherein said ionic filtration with potential feedback stage filter element is in combination with a voltage power supply which provides a potential difference between the ionizing element and the conducting collector element with minimum of −3000 volts.

6. The apparatus of claim 3, wherein said electrostatic filtration stage filter element comprises
an electrical coupling means for receiving an electric potential from a high voltage source;
a first conductor electrode, which is connected to the positively charged pole of the said electrical coupling means;
a second conductor electrode, which is connected to the negatively charged pole of the said electrical coupling means, produces an electrostatic field between the said first conductor electrode and the said second conductor electrode due to the production of said high potential gradient when supplied with charge from a high voltage source through said electrical coupling means.

7. The apparatus of claim 1, wherein said electronic filter element of a portable potential feedback human breath filtration device adapted to be carried on a human body is an ionic filtration stage with potential feedback filter element, which comprises
a conducting collector element;
an electrical coupling means for receiving an electric potential from a high voltage source;
an ionizing element comprising an electrically conductive material having pointed sharp ends for providing a high potential gradient to ionize particle components of a gas passing there-through, said conducting collector element and ionizing element being connected to the electrical coupling means to produce said high potential gradient when supplied with charge from a high voltage source through said electrical coupling means;
the distance between the said electrically conductive material having pointed sharp ends and the positively charged surface of the said pre-filter stage filter element is close enough to affect the increase of electrical power consumption of electrically operating the control unit by at least 5% as compare to when the said distance of the said electrically conductive material having pointed sharp ends and the said positively charged surface of the said pre-filter stage filter element is at 30 mm apart at the closest location.

8. The apparatus of claim 3, wherein said electrostatic filtration stage filter element comprises an electrical coupling means for receiving an electric potential from a high voltage source;
a first conductor electrode, which is connected to the positively charged pole of the said electrical coupling means;
a second conductor electrode, which is connected to the negatively charged pole of the said electrical coupling means, produces an electrostatic field between the said first conductor electrode and the said second conductor electrode due to the production of said high potential gradient when supplied with charge from a high voltage source through said electrical coupling means.

9. The apparatus of claim 1, wherein said mask module of a portable potential feedback human breath filtration device is adapted to eye protection goggles to cover the eye portion of the user and functions as a face mask.

10. The apparatus of claim 1, wherein said the said mask module of a portable potential feedback human breath filtration device is adapted to a hood covering the head and functions as a hood mask.

11. The apparatus of claim 1, wherein said the said mask module of a portable potential feedback human breath filtration device is adapted to a helmet covering the head and functions as a helmet with mask.

12. The apparatus of claim 3, wherein said ultraviolet ray (UV) generating device element is an UV ray generating source generates UV rays with wavelength between 200 nm to 400 nm for germicidal sterilization functions.

13. The apparatus of claim 12, wherein said ultraviolet ray (UV) generating source is an ultra violet ray generating light emitting diode (LED).

14. The apparatus of claim 3, wherein said direct air purge blockage cover is equipped with an ultraviolet ray (UV) generating source on the surface facing towards the said ionic filtration with potential feedback stage filter element.

15. The apparatus of claim 3, wherein said the rear anti-UV leakage cover system is equipped with an ultraviolet ray (UV) generating source on the surface facing towards the said ionic filtration with potential feedback stage filter element.

16. The apparatus of claim 3, wherein said ultraviolet ray (UV) reflection system is further comprised of
a reflective surface on inside facing toward the ionic filtration with potential feedback stage filter element of the said direct air purge blockage cover;
one or more reflective surface on the electrostatic filtration stage filter element;
a reflective surface on the inside facing toward the ionic filtration with potential feedback stage filter element of the rear anti-UV leakage cover system;
to allow the UV rays to reflect between the elements and radiate onto all the electrically charged surfaces of the said electrostatic filtration stage filter element.

17. The apparatus of claim 3, wherein said rear anti-UV leakage cover system provides resistance and blockage of flow of body fluid from the user's mouth and nose directly into the said ionic filtration with potential feedback stage filter element caused by sneezing, coughing, and speech by the user.

18. The apparatus of claim 1, wherein said an ionization flux sensing device is further comprised of
a housing;
a sensing antenna;
a sensor power switch;
a sensor electronic unit with battery;
a LED which lights up when power is ON;
a sensor flux strength LED.

19. The apparatus of claim 18, wherein said ionization flux sensing device is to be used to detect the presence of ions generated by ionization with the said sensor electronic unit with battery will magnify the electrical energy received at the said sensing antenna by more than 40,000 time to light up the said sensor flux strength LED.

20. The apparatus of claim 18, wherein said ionization flux sensing device is to be used to detect the presence of electrical flux field with the said sensor electronic unit with battery will magnify the electrical energy received at the said sensing antenna by more than 40,000 time to light up the said sensor flux strength LED.

21. The apparatus of claim 1, wherein said USB power conversion cable receives power from USB power source outlet installed in public transportation systems which also include airplane, train, bus, taxis, and ferry.

22. The method of providing electromechanical air purification system in the form of respirator mask to passenger by adapting the said respirator mask to the face of the passenger to filter particles, airborne matters, dust, pollens, odor, PM2.5, contaminants, bacteria, viruses, toxic chemical, fume and tobacco smoke from entering the said passenger's respiratory system in public transportation systems which also include train, airplane, bus, ferry and taxis with the power source from an electrical power outlet which also includes USB 5 VDC power outlet installed within 2 meters from the said passenger's seat.

23. The method of providing electromechanical air purification system in the form of head covering hood to passenger by adapting the said head covering hood to cover the head of the passenger to filter particles, airborne matters, dust, pollens, odor, PM2.5, contaminants, bacteria, viruses, toxic chemical, fume and tobacco smoke from entering the said passenger's respiratory system in public transportation systems which also include train, airplane, bus, ferry and taxis with the power source from an electrical power outlet which also includes USB 5 VDC power outlet installed within 2 meters from the said passenger's seat.
